(19) United States
(12) Patent Application Publication
(10) Pub. No.: US 2003/0143576 A1
(43) Pub. Date: Jul. 31, 2003

(54) Method and device for integrated protein expression, purification and detection

(76) Inventors: Heman Chao, Aurora (CA); Wah Y. Wong, Edmonton (CA); Baomin Tian, Edmonton (CA); Donald Segal, Stouffville (CA); Jerry McElroy, Richmond Hill (CA)

Correspondence Address: PERKINS COIE LLP, P.O. BOX 2168, MENLO PARK, CA, 94026, US

(21) Appl. No.: 10/225,788

(22) Filed: Aug. 22, 2002

Related U.S. Application Data
(60) Provisional application No. 60/314,333, filed on Aug. 22, 2001.
(60) Provisional application No. 60/375,627, filed on Apr. 25, 2002.

(51) Int. Cl. C12Q 1/68

(52) U.S. Cl. 435/6

(57) ABSTRACT

A method for presenting a target protein or an array of target proteins for analysis is disclosed. The method includes the steps of forming a coding sequence; placing the coding sequence and protein-synthesis components capable of expressing the target protein under selected protein-synthesis conditions in a well on a substrate, the well having a surface which has been functionalized with the second coil-forming peptide; expressing said coding sequence under such conditions, wherein the target protein so synthesized binds to the well through coil-coil heterodimer formation, and is thus presented for analysis in the well in captured form; and washing said well to remove unbound components. The coding sequence includes a first nucleic acid sequence which encodes a first coil-forming peptide having a selected charge and being capable of interacting with a second, oppositely charged coil-forming peptide to form a stable α-helical coiled-coil heterodimer; and a second nucleic acid sequence encoding the target protein. Also disclosed are kits for use in practicing the method.

This application claims the benefit of U.S. Provisional Application No. 60/314,333, filed Aug. 22, 2001 and U.S. Provisional Application No. 60/375,627, filed Apr. 25, 2002, both of which are incorporated herein by reference in their entireties.

### FIELD OF THE INVENTION

The present invention relates to methods for presenting a target protein or an array of target proteins for analysis, and to kits for use in practicing the invention.

### REFERENCES

Innis et al., PCR PROTOCOLS, Academic Press, Inc., San Diego Calif., (1990).

Krieg, P. A. and Melton, D. A., Meth. Enzymol. 155:397, (1984).

Leibowitz, et al., Methods in Enzymology 194:536-544 (1991).

Lesley et al., J. Biol. Chem. 266:2632 (1991).

Melton, D. A., et al., Nucl. Acids Res. 12:7035, (1984).

Tripet, B., et al., Prot. Eng. 9:1029, (1996).

### BACKGROUND OF THE INVENTION

Proteins are the major components of cells. They determine the shape, structure, and function of the cell. Proteins are assembled by twenty different amino acids each with a distinct chemical property. This variety allows for enormous versatility in the chemical and biological properties of different proteins. Despite the fact that new proteins are being discovered at an unprecedented rate, protein structure and function studies are lagging behind, mainly due to a lack of high throughput methods.

It is often necessary to immobilize and present proteins on a solid support during the process of studying proteins. For example, in Western blot analysis and phage display screening of protein expression libraries proteins are immobilized non-covalently. In some other applications such as immunoprecipitation and affinity purification, agents (e.g., antibodies and ligands) are covalently conjugated onto solid supports (e.g., agarose beads) through their primary amines, sulfhydryls or other reactive groups. Although there are a number of methods for immobilizing peptides and proteins directly to solid supports, these bound proteins sometimes lose their ability to interact with other proteins orligands after immobilization.

Therefore, there is a need for improved techniques for presenting proteins on solid supports which retain their ability to interact with other proteins. The method should allow rapid and detailed analysis of multiple proteins for both basic research and clinical medicine. Such techniques would be extremely valuable in monitoring protein-protein, protein-small molecule, and protein-nucleic acid interactions under an array of different conditions. The present invention is designed to meet these needs.

### SUMMARY OF THE INVENTION

Accordingly, it is an object of the invention to provide, in one aspect, a method for presenting a target protein for solid-phase analysis. The steps involved in practicing the method include forming a mixture in a well on a substrate. The mixture includes a coding sequence that includes a first nucleic acid sequence which encodes a first coil-forming peptide having a selected charge and being capable of interacting with a second, oppositely charged coil-forming peptide to form a stable α-helical coiled-coil heterodimer; and a second nucleic acid sequence encoding the target protein. The mixture also includes protein-synthesis components capable of expressing the target protein under selected protein-synthesis conditions in the well. The well has a surface which has been functionalized with the second coil-forming peptide. The mixture is allowed to react under conditions such that the target protein is synthesized and binds to the well through coil-coil heterodimer formation, and is thus presented for analysis in the well in captured form. The well is then washed to remove unbound components.

In one embodiment, the coding sequence is formed by cloning the second nucleic acid sequence into a cleavable site of a cloning vector containing the first nucleic acid sequence such that the first nucleic acid sequence is in frame with the second nucleic acid sequence.

In another embodiment, the cloning vector includes the following components in the 5′ to 3′ direction and operably linked: a transcription and translation initiation region; the cleavable site at which a nucleic acid encoding the target protein can be inserted; the first nucleic acid sequence; and a transcription and translation termination region. Alternatively, the first nucleic acid sequence resides upstream of the cleavable site.

In yet another embodiment, forming the coding sequence includes the steps of ligating the first nucleic acid sequence to the second nucleic acid sequence to form a chimeric coding sequence, and amplifying the chimeric coding sequence with PCR primers designed to hybridize with and amplify the chimeric coding sequence. Alternatively, forming the coding sequence may include the steps of: optionally decapping the second nucleic acid sequence, where the second nucleic acid sequence is a mRNA molecule; ligating, to a 5′ end of the mRNA molecule to form a RNA template, a first oligonucleotide primer that includes the first nucleic acid sequence which encodes the first coil-forming peptide, and a transcription initiation region which is oriented to transcribe towards the 3′ end; reverse transcribing the RNA template with reverse transcriptase, deoxyribonucleotide triphosphates and a second oligonucleotide primer comprising an oligonucleotide dT sequence to form first strand cDNA; removing the mRNA from the first strand cDNA; and incubating the first strand cDNA, a DNA polymerase, deoxyribonucleotide triphosphates, and a third oligonucleotide primer comprising at least 12 nucleotides of the first primer sequence, to form double stranded cDNA; amplifying the double stranded cDNA with DNA polymerase, deoxyribonucleotide triphosphates, a fourth oligonucleotide primer complementary to at least 12 nucleotides of the 3′ end of a first strand of the cDNA, and a fifth oligonucleotide primer complementary to at least 12 nucleotides of the 3′ end of the second strand. Forming the coding sequence may include: optionally decapping said second nucleic acid sequence, where said second nucleic acid sequence is a mRNA molecule; ligating a first oligonucleotide primer to a 5′ end of the mRNA molecule to form a RNA template, reverse transcribing the RNA template with reverse transcriptase, deoxyribonucleotide triphosphates and a first oligonucleotide primer comprising an oligonucleotide dT sequence to form first strand cDNA; removing the mRNA from the first strand cDNA; incubating the first strand cDNA, a DNA polymerase, deoxyribonucleotide triphosphates, and a second oligonucleotide primer comprising at least 12 nucleotides of the first primer sequence, to form double stranded cDNA, amplifying the double stranded cDNA with DNA polymerase, deoxyribonucleotide triphosphates, a third oligonucleotide primer including a region complementary to at least 12 nucleotides of the 3′ end of a first strand of the cDNA, and a restriction enzyme site compatible with a first restriction enzyme site in a cloning vector; and a fourth oligonucleotide primer that includes a region complementary to at least 12 nucleotides of the 3′ end of the second strand, and a restriction enzyme site compatible with a second restriction enzyme site in the cloning vector; digesting the amplification product and the cloning vector with restriction enzymes capable of cutting at the first and second restriction enzyme sites; and cloning the digested amplification product into the cloning vector.

The amplification product may be translated in vitro by further including the steps of transcribing the template sequence in vitro using a DNA-dependent RNA polymerase that recognizes the transcription initiation region in said amplification product, and combining the transcription products with an appropriate cell free in vitro translation system. The nucleic acid sequence of interest may be translated in vitro, by further including the steps of: linearizing the cloning vector with a restriction enzyme that cleaves downstream from the coding sequence; transcribing the template sequence in vitro using a DNA-dependent RNA polymerase that recognizes the transcription initiation region in the cloning vector; and combining the transcription products with an appropriate cell free in vitro translation system.

In one embodiment, the placing includes transforming or transfecting the coding sequence into cells capable of translating the coding sequence, where the protein-synthesis components include the cells.

In another aspect, the invention contemplates a method for carrying out the presentation of a plurality of target proteins. The steps in performing the method include: adding to each of a plurality of wells in a substrate, each well having a first coil-forming peptide therein, a selected one of a plurality of different-sequence nucleic acid molecules, each having a common-sequence capture portion encoding a second coil-forming peptide and a different-sequence target portion encoding a target protein; filling said wells with a solution comprising protein synthesis components capable of expressing the different-sequence nucleic acid molecules under selected protein-synthesis conditions; promoting expression of the different-sequence nucleic acid molecules under such conditions, wherein the target protein expressed in each well binds to the well through coil-coil heterodimer formation and is thus presented for analysis in the well in captured form; and washing the wells to remove unbound components.

In one embodiment, the substrate is an array of 96 wells. In another embodiment, the substrate is a MALDI-MS plate having wells capable of holding said solution.

In yet another aspect, the invention includes a kit for presenting one or more target proteins for solid-phase analysis for use with a cell free in vitro translation system. The kit includes a substrate containing a plurality of wells, wherein each well is functionalized with a first coil-forming peptide having a selected charge and being capable of interacting with a second, oppositely charged coil-forming peptide to form a stable α-helical coiled-coil heterodimer; a cloning vector comprising in the 5′ to 3′ direction and operably linked (i) a transcription and translation initiation region, (ii) a nucleic acid sequence which encodes said second coil-forming peptide, (iii) a transcription and translation termination region. The vector also has a cleavable site at which a nucleic acid encoding the target protein can be inserted between (i) and (ii) or between (ii) and (iii).

In still another aspect, the invention includes a multiplexed in vitro cell free protein synthesis system. The system includes a substrate that includes a plurality of wells, each well having bound thereto a first coil-forming peptide having a selected charge and being capable of interacting with a second, oppositely charged coil-forming peptide to form a stable α-helical coiled-coil heterodimer. In each of the wells is contained (i) a coding sequence that includes (A) a first nucleic acid sequence which encodes a first coil-forming peptide having a selected charge and being capable of interacting with a second, oppositely charged coil-forming peptide to form a stable α-helical coiled-coil heterodimer; and (B) a second nucleic acid sequence encoding the target protein. Each of the wells also contains protein-synthesis components capable of expressing the target protein under selected protein-synthesis conditions in the well, said well having a surface which has been functionalized with the second coil-forming peptide. The mixture reacts under conditions such that the target proteins are synthesized and bind to the well through coil-coil heterodimer formation, and are thus presented for analysis in each of the wells in captured form.

These and other objects and features of the invention will be more fully appreciated when the following detailed description of the invention is read in conjunction with the accompanying drawings.

### BRIEF DESCRIPTION OF THE DRAWINGS

FIG. 1 is a perspective view of a target protein presentation kit constructed in accordance with one embodiment of the present invention;

FIG. 2 is a cross-sectional view taken in the direction of arrows 2-2 in FIG. 1 of a presentation kit containing a different target protein in each well constructed in accordance with one embodiment of the invention;

FIG. 3A is a map showing the features and relevant restriction sites of plasmid pET-17b containing a sequence of interest and C-terminal coiled-coil domain;

FIG. 3B is a map showing the features and relevant restriction sites of plasmid pET-17b containing a sequence of interest and N-terminal coiled-coil domain;

FIGS. 4A-4E show steps in in vitro synthesis of the coding sequence in one embodiment of the present invention;

FIGS. 5A-5C illustrate steps in anchoring a primer to mRNA and producing cDNA in one embodiment of the present invention;

FIGS. 6A-6C show steps in producing and anchoring a target protein to a substrate in accordance with one embodiment of the present invention;

FIGS. 7A-7C illustrate steps in in vitro translating and anchoring a target protein to a well in a substrate in accordance with one embodiment of the present invention;

FIG. 8 is a cross-sectional view taken in the direction of arrows 2-2 in FIG. 1 of a presentation kit containing a different mutated target protein in each well;

FIG. 9 is a cross-sectional view taken in the direction of arrows 2-2 in FIG. 1 of a presentation kit containing the same target protein in each well.

FIGS. 10A-10C show the mass spectrum of Tnl peptide-E coil protein (pMA2) expressed using commercial extract and captured by K coil-immobilized surface, and appropriate controls.

FIGS. 11A-11C show the mass spectrum of Actin peptide-E coil protein (pS1A) expressed using commercial extract and captured by K coil-immobilized surface, and appropriate controls.

FIGS. 12A-12C show the mass spectrum of cMyc peptide-E coil protein (cMyc-E) expressed using commercial extract and captured by K coil-immobilized surface, and appropriate controls.

FIGS. 13A-13C show the mass spectrum of cMyc peptide-E coil protein (cMyc-E) expressed using in-house extract and captured by K coil-immobilized surface, and appropriate controls.

### DETAILED DESCRIPTION OF THE INVENTION

I. Definitions

Unless otherwise indicated, all technical and scientific terms used herein have the same meaning as they would to one skilled in the art of the present invention. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

All publications and patents cited herein are expressly incorporated herein by reference for the purpose of describing and disclosing compositions and methodologies which might be used in connection with the invention.

The term “support” as used herein refers to the materials on which agents are deposited and immobilized.

The term “peptide” as used herein refers to a compound made up of a single chain of amino acid residues linked by peptide bonds. The term “protein” as used herein may be synonymous with the term “peptide” or may refer, in addition, to a complex of two or more peptides.

The term “nucleic acid molecule” includes RNA, DNA and cDNA molecules. It will be understood that, as a result of the degeneracy of the genetic code, a multitude of nucleotide sequences encoding given peptides such as E-coil and K-coil peptides may be produced.

A “heterologous” nucleic acid construct or sequence has a portion of the sequence which is not native to the cell in which it is expressed. Heterologous, with respect to a control sequence refers to a control sequence (i.e. promoter or enhancer) that does not function in nature to regulate the same gene the expression of which it is currently regulating. Generally, heterologous nucleic acid sequences are not endogenous to the cell or part of the genome in which they are present, and have been added to the cell, by infection, transfection, microinjection, electroporation, or the like. A “heterologous” nucleic acid construct may contain a control sequence/DNA coding sequence combination that is the same as, or different from a control sequence/DNA coding sequence combination found in the native cell.

As used herein, the term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art.

As used herein, an “expression cassette” or “expression vector” is a nucleic acid construct generated recombinantly or synthetically, with a series of specified nucleic acid elements that permit transcription of a particular nucleic acid in a target cell or in vitro. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter.

As used herein, the term “plasmid” refers to a circular double-stranded (ds) DNA construct used as a cloning vector, and which forms an extrachromosomal self-replicating genetic element in many bacteria and some eukaryotes.

As used herein, the term “selectable marker-encoding nucleotide sequence” refers to a nucleotide sequence which is capable of expression in host cells and where expression of the selectable marker confers to cells containing the expressed gene the ability to grow in the presence of a corresponding selective agent.

As used herein, the terms “promoter” and “transcription initiator” refer to a nucleic acid sequence that functions to direct transcription of a downstream gene. The promoter will generally be appropriate to the host cell in which the target gene is being expressed. The promoter together with other transcriptional and translational regulatory nucleic acid sequences (also termed “control sequences”) are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

“Chimeric gene” or “heterologous nucleic acid construct”, as defined herein refers to a non-native gene (i.e., one that has been introduced into a host) that may be composed of parts of different genes, including regulatory elements. A chimeric gene construct for transformation of a host cell is typically composed of a transcriptional regulatory region (promoter) operably linked to a heterologous protein coding sequence, or, in a selectable marker chimeric gene, to a selectable marker gene encoding a protein conferring antibiotic resistance to transformed host cells. A typical chimeric gene of the present invention, for transformation into a host cell, includes a transcriptional regulatory region that is constitutive or inducible, a protein coding sequence, and a terminator sequence. A chimeric gene construct may also include a second DNA sequence encoding a signal peptide if secretion of the target protein is desired.

A nucleic acid is “operably linked” when it is placed into a functional relationship with another nucleic acid sequence. For example, DNA encoding a secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site isoperably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “gene” means the segment of DNA involved in producing a polypeptide chain, that may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention.

The term “introduced” in the context of inserting a nucleic acid sequence into a cell, means “transfection”, or “transformation” or “transduction” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

As used herein, the term “expression” refers to the process by which a polypeptide is produced based on the nucleic acid sequence of a gene. The process includes both transcription and translation.

The term “signal sequence” refers to a sequence of amino acids at the N-terminal portion of a protein which facilitates the secretion of the mature form of the protein outside the cell. The mature form of the extracellular protein lacks the signal sequence which is cleaved off during the secretion process.

By the term “host cell” is meant a cell that contains a vector and supports the replication, or transcription and translation (expression) of the expression construct. Host cells for use in the present invention can be prokaryotic cells, such as E. coli, or eukaryotic cells such as yeast, plant, insect, amphibian, or mammalian cells.

As used herein, the terms “active” and “biologically active” refer to a biological activity associated with a particular target protein, such as the enzymatic activity. It follows that the biological activity of a given protein refers to any biological activity typically attributed to that protein by those of skill in the art.

II. Kit

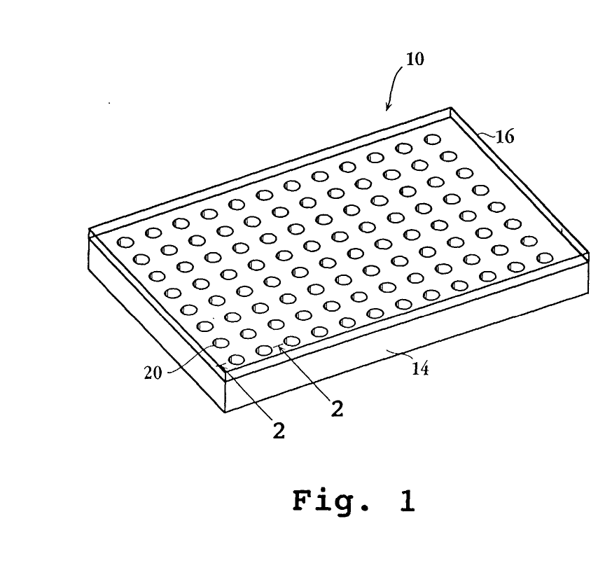

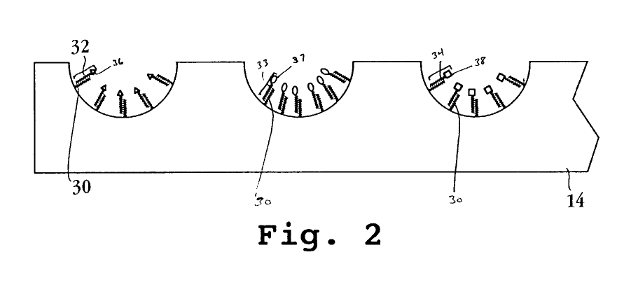

As a matter of convenience, a substrate and predetermined amounts of reagents employed in the present invention can be provided in a kit in packaged combination. Hence, in one aspect, the invention provides a kit for carrying out the presentation of a plurality of target proteins. FIG. 1 is a plan view of a kit 10 that includes a substrate 14 and, optionally, a covering 16 which can be transparent and is attached to the substrate. The substrate includes a plurality of discrete wells 20. As shown in FIG. 2, each well 20 in substrate 14 is functionalized with a first coil-forming peptide 30 having a selected charge and being capable of interacting with a second, oppositely charged coil-forming peptide to form a stable α-helical coiled-coil heterodimer. The two oppositely charged peptides spontaneously self-assemble into a heterodimer complex. The interaction of coiled-coil heterodimers is further described in Section IV below.

Any compatible substrate can be used in conjunction with this invention. The substrate (usually a solid) can be any of a variety of organic or inorganic materials or combinations thereof, including, merely by way of example, plastics such as polypropylene or polystyrene; ceramic; silicon; (fused) silica, quartz or glass, which can have the thickness of, for example, a glass microscope slide or a glass cover slip; paper, such as filter paper; diazotized cellulose; nitrocellulose filters; nylon membrane; or polyacrylamide or other type of gel pad, e.g., an aeropad or aerobead, made of an aerogel, which is, e.g., a highly porous solid, including a film, which is prepared by drying of a wet gel by any of a variety of routine, conventional methods. Substrates that are transparent to light are useful when the method of performing an assay involves optical detection. In a preferred embodiment, the substrate is the plastic surface of a multiwell, e.g., tissue culture plate, for example a 24-, 96, 256-, 384-, 864- or 1536-well plate. In another preferred embodiment, the substrate is a plate suitable for use in a Matrix Assisted Laser Desorption Ionization-Time of Flight mass spectrometer (MALDI-MS). An exemplary method of performing MALDI-MS is described in U.S. Pat. No. 6,111,251 which is expressly incorporated by reference in its entirety herein. Additional substrates and methods for binding and identifying proteins may be found in U.S. Pat. Nos. 6,027,942 and 5,719,060, both of which are incorporated by reference herein in their entireties.

MALDI-MS has become established as a method for mass determination of biopolymers and substances such as peptides, proteins and DNA fragments. In this method, the substance to be analyzed is typically placed in a solution of matrix material and coated onto a support or substrate. The solute evaporates, leaving the analyte in a solid matrix which is then illuminated to cause the analyte molecules or synthetic polymers to be desorbed. This desorption process is especially useful for releasing large biological molecules without charring, fragmentation or chemical degradation to a mass spectrometer or similar instrument for separation and detection.

The substrate comprises regions which are spatially discrete and addressable or identifiable. Each region comprises a coiled-coil peptide 30 bound thereto. In one embodiment, the regions can be separated from one another by any physical barrier which is resistant to the passage of liquids. In another embodiment, a substrate such as a MALDI-MS plate can be etched out to have discrete, shallow wells. Alternatively, a substrate can comprise regions with no separations or wells, for example a flat surface, and individual regions can be further defined by overlaying a structure (e.g., a piece of plastic or glass) which delineates the separate regions. The relative orientation of the regions can take any of a variety of forms including, but not limited to, parallel or perpendicular arrays within a square or rectangle or other surface, radially extending arrays within a circular substrate, or linear arrays. The number of bound coil-forming peptides in a region can be one, or preferably at least two. In one embodiment, the density of the bound coil-forming peptides in a region is between about 1×10² to about 1×10¹⁵ molecules/mm², preferably between about 1×10⁴ to about 1×10¹² molecules/mm², more preferably between about 1×10⁶ to about 1×10¹⁰ molecules/mm², and most preferably about 8.5×10¹¹ molecules/mm².

The kit may also include a cloning vector that contains a nucleic acid sequence which encodes the second coil-forming peptide, as described in Section IIIA below. Furthermore, the kit may contain an in vitro translation system as described in greater detail in Section IIIC below. The kit may also include various buffered media, some of which may contain one or more of the above components.

The relative amounts of the various reagents in the kit can be varied widely to provide for concentrations of the reagents necessary to carry out the protein presentation methodology of the present invention. Under appropriate circumstances one or more of the reagents in the kit can be provided as a dry powder, usually lyophilized, including excipients, which on dissolution will provide for a reagent solution having the appropriate concentrations for performing a method or assay in accordance with the present invention. Each reagent can be packaged in separate containers or some reagents can be combined in one container where cross-reactivity and shelf life permit.

The kit may also include a written description of a method in accordance with the present invention as described above. The instructions can include, for example, a description of the first coil-forming peptides on the surface, an indication of how many peptides there are and where on the surface they are located. The instructions can also include a protocol for associating the bound peptides and expressed proteins, e.g., conditions and reagents for in vitro translation, temperature and time of incubation, and conditions and reagents for removing unassociated molecules (e.g. washes), and the like. Furthermore, the instructions can encompass any of the parameters, conditions or embodiments disclosed in this application.

III. Forming and Expressing the Coding Sequence

A. Vector Construction

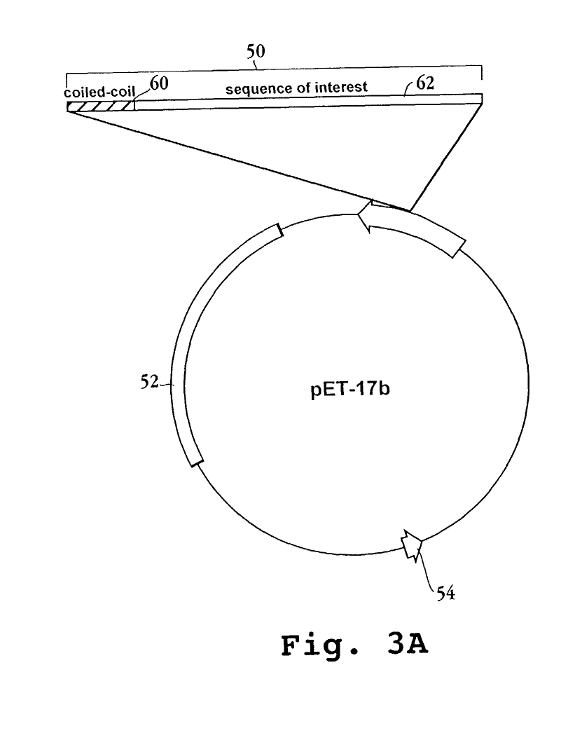

FIGS. 3A and 3B illustrate expression vectors for use in the present invention comprising a coding sequence 50 (or expression cassette), designed for operation in an in vitro or in vivo expression system, with companion sequences 52 and 54 upstream and downstream from the coding sequence. The coding sequence may have the coiled-coil region 60 at the C-terminus of the sequence of interest 62 as shown in FIG. 3A. Alternatively, the coiled-coil region 60 of the coding sequence 50 may be at the N-terminus of the sequence of interest 62, as shown in FIG. 3B.

The companion sequences will be of plasmid or viral origin and provide the necessary characteristics to the vector to permit the vectors to be replicated in a host cell. Suitable transformation vectors are described below. Suitable components of the expression plasmid, including a trancription and translation initiator, a coding sequence encoding the protein of interest and coiled-coil region, and suitable transcription and translation terminators are also discussed below. Three exemplary plasmids are the pET-17b[pMA2], pET-17b[pS1A], and pET-17b[cMyc-E] plasmids.

A1. Transcription & Translation Initiators

The transcription initiators of the present invention can be any sequence capable of initiating transcription of a coding sequence in an in vitro or in vivo context. Thus, the transcription initiator will generally need to have available the RNA polymerase enzyme appropriate for in vitro transcription from that transcription initiator sequence. The transcription initiator can be, for example, a T3 or SP6 promoter. These promoters are used in conjunction with the corresponding T3, and SP6 RNA polymerases for making the mRNA from a double stranded linear DNA template in vitro. Preferably, the transcription initiator is the T7 promoter (SEQ ID NO: 5), which is used in conjunction with the T7 transcription terminator as described below. The promoter may be a natural sequence or alternatively a synthetic sequence. In double stranded DNA the transcription initiator is upstream of a coding sequence oriented to transcribe downstream.

A2. Transcription and Translation Terminators

The termination regulatory region of the expression cassette may be native with the transcription initiation region, or may be derived from another source. The transcriptional termination region may be selected, particularly for stability of the mRNA, to enhance expression. Preferably the transcription terminator is the T7 transcription terminator (SEQ ID NO: 6).

A3. Construction of the Vector

The nucleotide sequences of the present invention are useful for producing hybrid coiled-coil regions attached to proteins of interest in an in vitro expression system. In this manner, the nucleotide sequences encoding the hybrid polypeptides of the invention are provided in expression cassettes. Such an expression cassette may be provided with a plurality of restriction sites for insertion of the nucleotide sequence to be under the transcriptional regulation of the regulatory regions.

As described above, the coiled-coil region can be at either the C-terminal end or the N-terminal end of the protein of interest. As is the case for the protein of interest, each of the other elements present in the hybrid polypeptide can be a known naturally occurring polypeptide sequence or can be synthetically derived, including any variants thereof that do not adversely affect the function of the hybrid polypeptide as described herein. By “adversely affect” is intended that inclusion of the variant form of the element results in decreased bioactivity of the hybrid polypeptide relative to the hybrid polypeptide comprising the native form of the element.

In preparing the expression cassette, the various nucleotide sequence fragments may be manipulated so as to provide for the sequences in the proper orientation and, as appropriate, in the proper reading frame. Toward this end, adapters or linkers may be employed to join the nucleotide fragments or other manipulations may be involved to provide for convenient restriction sites, removal of superfluous nucleotides, removal of restriction sites, or the like. For this purpose, in vitro mutagenesis, primer repair, restriction, annealing, resubstitutions, e.g., transitions and transversions, may be involved. See particularly Sambrook et al. (1989).

The expression cassettes of the present invention can be ligated into a replicon (e.g., plasmid, cosmid, virus, mini-chromosome), thus forming an expression vector that is capable of autonomous DNA replication in vivo. Preferably the replicon will be a plasmid. Such a plasmid expression vector will be maintained in one or more replication systems that allow for stable maintenance within a prokaryotic host for cloning purposes.

FIGS. 3A and 3B show an exemplary tranformation vector for use in expressing the hybrid polypeptide in an in vitro translation system. Details of the vector construction are given in Example 1B.

B. Forming the PCR Product

The coding sequence can be formed by ligating a first nucleic acid sequence to a second nucleic acid sequence to form a chimeric coding sequence, and amplifying the chimeric coding sequence with PCR primers designed to hybridize with and amplify the chimeric coding sequence. The first nucleic acid sequence should encode a first coil-forming peptide which has a selected charge and is capable of interacting with a second, oppositely charged coil-forming peptide to form a stable α-helical coiled-coil heterodimer. The second nucleic acid sequence encodes the target protein of interest.

The design of PCR primers that hybridize with selected sequences is known in the art. See, for example, U.S. Pat. Nos. 4,683,195, 4,683, 202, and 4,800,159; andInnis et al., 1990, each of which is incorporated herein by reference in its entirety.

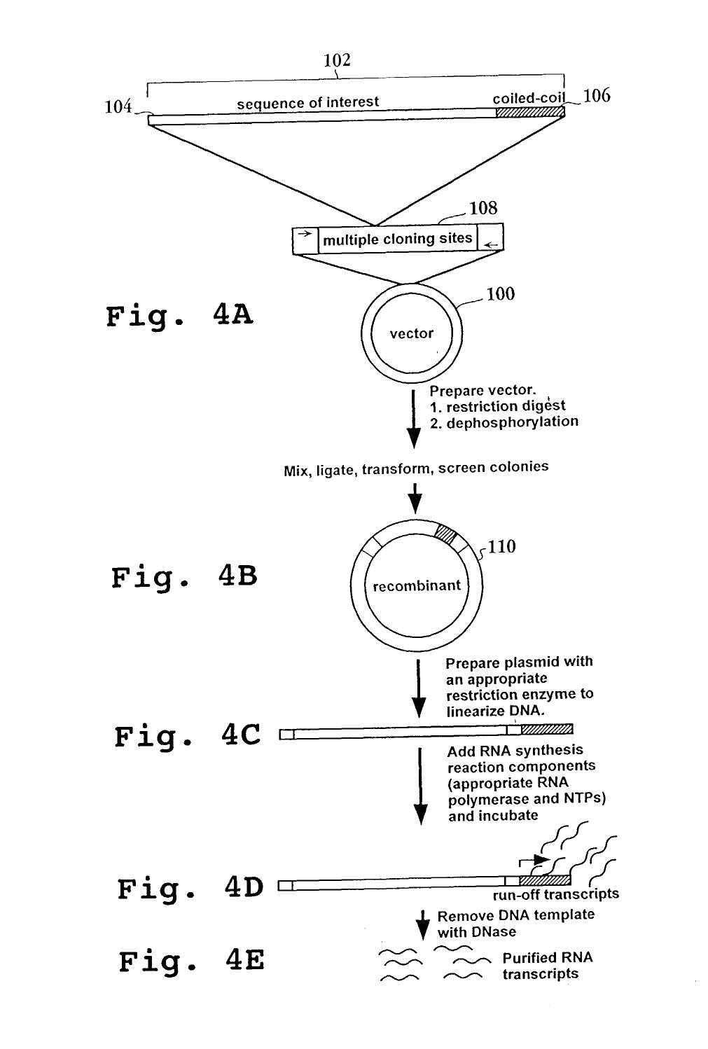

An alternative method of the invention for forming the coding sequence is to isolate mRNA from cells or tissues. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. mRNA molecules comprise a nucleotide sequence ending in a poly A tail, and are single stranded. As illustrated in FIGS. 5A and 5B, the mRNA 155 may be decapped by techniques standard in the art. For example, enzymes and reagents can be purchased commercially from Epicentre Technologies in Madison, Wis., including tobacco acid pyrophosphatase for decapping RNA. A first oligonucleotide primer 150 is ligated to the 5′ end of the mRNA with a ligase capable of ligating single stranded RNA to single stranded DNA, for example T4 RNA ligase. The first primer comprises a transcription initiation region which is oriented to transcribe towards the 3′ end of the mRNA. In one embodiment, the first primer also comprises a first nucleic acid sequence which encodes a first coil-forming peptide. As discussed above, the transcription initiation region can be any transcription initiation sequence capable of facilitating in vitro transcription, including, for example a T7, T3, or SP6 promoter sequence. Generally, these promoters will be paired with the appropriate RNA polymerase enzyme, which accomplishes the in vitro transcription.

A second primer 160 having an oligonucleotide dT sequence is added to the reaction with reverse transcriptase enzyme and appropriate buffers and deoxyribonucleotidetriphosphates to achieve reverse transcription of the sense mRNA strand. In one embodiment, the second primer has an oligonucleotide dT sequence of at least 10 consecutive dTs. The mRNA can then be removed by any appropriate means, including, for example, addition of NaOH or RNase.

At this stage single stranded cDNA has been made and includes from 5′ to 3′ the second primer sequence, and the complementary sequence to the mRNA and first primer. The first primer 150, or at least a 12 nucleotide sequence of the first primer can then be used in the reaction to generate double stranded cDNA 165, shown in FIG. 5C, from the single stranded antisense strand, using DNA polymerase and deoxyribonucleotidetriphosphates. From the double stranded cDNA, cRNA can be generated in the presence of RNA polymerase in an in vitro reaction. The RNA polymerase will be appropriate for the promoter sequence. For example, where a T7 promoter is used, a T7 RNA polymerase is used to catalyze the in vitro transcription reaction. The cDNA can also be amplified using a 5′ and a 3′ primer, each of which is complementary to a different strand. Each amplification primer may contain a restriction enzyme site compatible with a first restriction enzyme site in a cloning vector that may contain a N-terminal or C-terminal coiled-coil peptide encoding DNA sequence. The amplification is carried out using a DNA polymerase, for example Taq DNA polymerase, deoxyribonucleotidetriphosphates, and appropriate buffer and termperature conditions for polymerase chain reaction (PCR).

The cDNA can also be ligated into a vector for performing other manipulations, including expression, or other amplifications or analysis. From the linear double stranded cDNA the coding sequence can be in vitro transcribed and translated. Vectors for expression can include any eukaryotic or bacterial expression vector, including mammalian, yeast, amphibian or insect expression vectors. The cDNA can be sequenced from the linear template, or placed in a sequencing vector.

C. In vitro RNA and Protein Synthesis

C1. RNA Synthesis

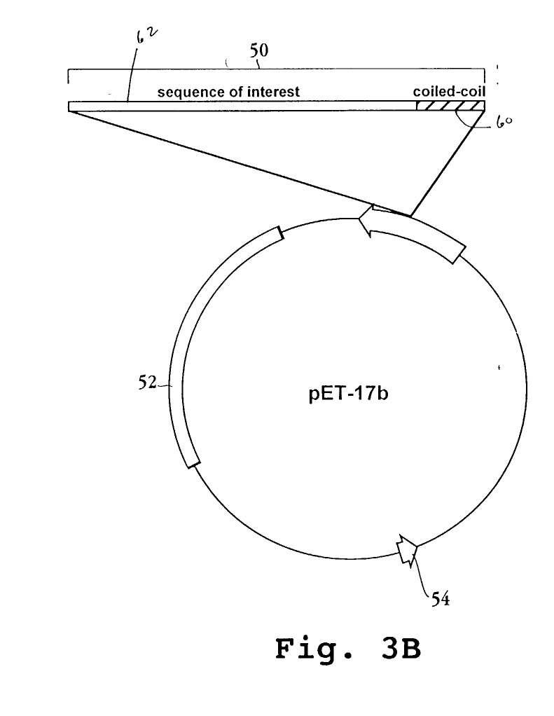

The coding sequence, as a PCR amplification product or cloned into a vector as described above, may be translated in vitro by transcribing the template sequence in vitro using a DNA-dependent RNA polymerase that recognizes the transcription initiation region, and appropriate components. FIGS. 4A-4E illustrate steps involved in the use of the present invention to create RNA transcripts in vitro. The figures show a plasmid 110 like the one shown in FIGS. 3A and 3B. The vector has a chimeric gene 102 that includes a sequence of interest region 104 and a coiled-coil region 106 inserted into the vector 100 in the multiple cloning region 108. If desired, the plasmid may be linearized with an appropriate restriction enzyme that cleaves downstream from the coding sequence prior to transcription as shown in FIG. 4C. Alternatively, when the presence of vector sequences on the probe will not interfere with subsequent applications, transcripts can be synthesized using an intact plasmid as the template.

If desired, linearized or intact plasmid DNA can be extracted with phenol:chloroform:isoamyl alcohol, ethanol precipitated, and suspended in TE or water before using the DNA for in vitro transcription reactions. RNA synthesis reaction components, including an appropriate RNA polymerase and NTPs, are incubated with the linearized or intact plasmid DNA to produce RNA transcripts. The DNA template can be removed with RNase-free DNase I to create purified RNA transcripts as in FIG. 4E. An exemplary method for RNA synthesis in vitro is described in Melton, 1984.

C2. Protein Synthesis

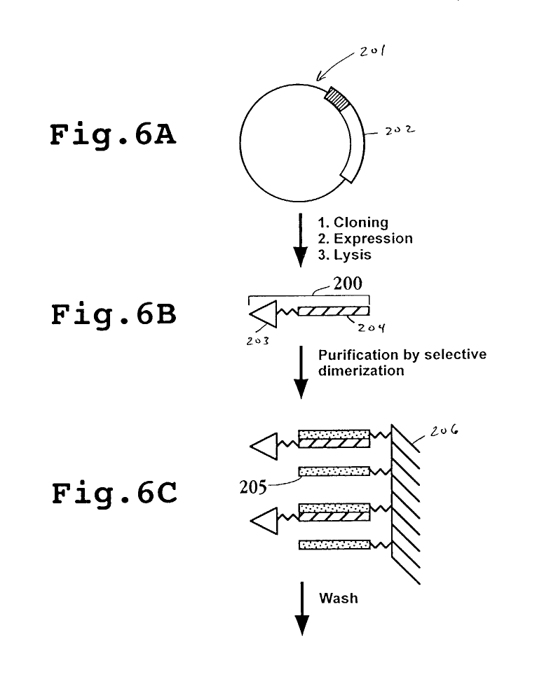

Following RNA synthesis, the transcription product 170 can be combined with an appropriate in vitro translation system to produce a target protein that includes a coiled-coil region on either the N-terminal or C-terminal end, as illustrated in FIGS. 7A-7B. Target proteins 175 produced by the in vitro translation system are capable of binding to the substrate 180 through the substrate-bound coiled-coil peptide 185. Exemplary methods for performing in vitro protein synthesis are described in Leibowitz, et al. (1991), Lesley et al. (1991); and U.S. Pat. Nos. 5,968,767 and 6,322,970, each of which is expressly incorporated by reference herein in its entirety.

If desired, the synthesized RNA molecules may be capped prior to translation. Capped RNA molecules synthesized in vitro are effective templates for translation. See, e.g., Krieg and Melton, 1984. Systems and protocols effective for capping synthesized transcripts prior to translation are available from commercial vendors such as Promega, WI; www.promega.com.

D. Protein Synthesis in Host Cells

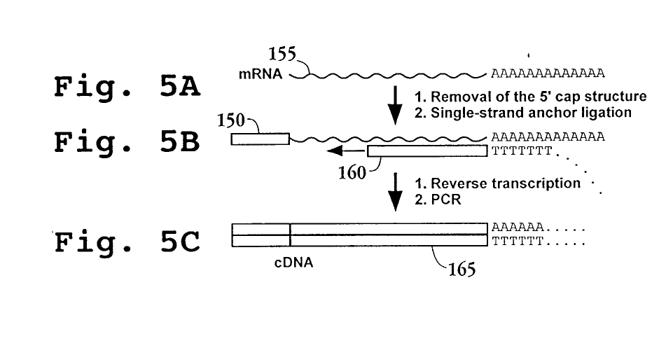

As an alternative to in vitro translation, the coding sequence 202, contained in an appropriate vector 201 as previously described, and illustrated in FIG. 6A, may be transformed or tranfected into a host cell for expression of the protein of interest 204 with the coiled-coil domain 203. The host cell may be placed in the wells of a substrate 206 prior to or following transformation or transfection, and subjected to conditions effective to express the protein of interest with the coiled-coil domain. The expressed hybrid polypeptide 200 may be secreted from the host cell and bind to the coiled-coil peptides 205 in each well. If the hybrid polypeptide 200 is not secreted, the host cells may belysed so that the protein 200 is released from the cell and capable of binding to the surface-bound coiled-coil peptides 205. The wells can then be washed to remove unbound components. Exemplary methods of expressing proteins and lysing cells are described in U.S. Pat. Nos. 6,238,861 and 5,496,549, both of which are expressly incorporated by reference herein in their entireties.

Host cells are transformed with expression constructs described above using a variety of standard techniques including, but not limited to,electroporation, microparticle bombardment, spheroplast generation methods, or whole cell methods such as those involving lithium chloride and polyethylene glycol (Cregg et al., 1985; Liu et al., 1992; Waterham et al., 1996; and Cregg and Russell, 1998).

Although not necessary for the basic method of the invention, promoter sequences, such as promoters that function in eukaryotic cell systems, such as yeast, mammalian, or insect promoters can be included in the vector sequence for facilitating expression in these cell systems. These additional promoter sequences which are for expression or other purposes may be distinguished from the promoter used for cell-free in vitro transcription, which was described above.

In one embodiment, a recombinant expression vector is engineered to contain a copy of the nucleic acid sequence encoding the E peptide (SEQ ID NO: 1) at the C-terminus or N-terminus of the sequence of interest. The plasmid may contain an inducible promoter, such as the IPTG inducible promoter, for selective hybrid polypeptide expression, a multiple cloning site for gene insertion, an ampicillin resistance gene for clone selection and a signal sequence to direct the newly made hybrid polypeptide to the periplasmic space to simplify purification. An exemplary method of engineering a coding sequence to include the nucleic acid sequence encdoding the E peptide at the C-terminus of a sequence of interest is described in Tripet, et al. 1996. Alternatively, the expression vector may contain the K peptide (SEQ ID NO: 2) or a variant thereof at the C-terminus or N-terminus of the sequence of interest. Additional coiled-coil peptides useful in the present invention may be found in U.S. Pat. Nos. 6,165,335, 6,130,037, 5,955,379, and 5,824,483, each of which is incorporated by reference in its entirety herein.

IV. Formation of the α-helical Coiled-Coil Complex

When a first coil-forming peptide and a second coil-forming peptide are mixed together under conditions favoring the formation of α-helical coiled-coil heterodimers, they interact to form a two-subunit α-helical coiled-coil heterodimeric complex. Peptides in an α-helical coiled-coil conformation interact with one another in a characteristic manner that is determined by the primary sequence of each peptide. The tertiary structure of an α-helix is such that seven amino acid residues in the primary sequence correspond to approximately two turns of the α-helix. Accordingly, a primary amino acid sequence giving rise to an α-helical conformation may be broken down into units of seven residues each, termed heptads. The heterodimer-subunit peptides are composed of a series of heptads in tandem. When the sequence of a heptad is repeated in a particular heterodimer-subunit peptide, the heptad may be referred to as a “heptad repeat”, or simply “repeat”.

A first coil-forming peptide and second coil-forming peptide may assemble into a heterodimer coiled-coil helix (coiled-coil heterodimer) in either parallel or antiparallel configurations. In a parallel configuration, the two heterodimer-subunit peptide helixes are aligned such that they have the same orientation (amino-terminal to carboxyl-terminal). In an antiparallel configuration, the helixes are arranged such that the amino-terminal end of one helix is aligned with the carboxyl-terminal end of the other helix, and vice versa.

Such heterodimer subunits are described in PCT patent application WO 95/31480 “Heterodimer Polypeptide Immunogen Carrier Composition and Method”, publication date Nov. 23, 1995, which is incorporated herein by reference in its entirety. Exemplary subunits are referred to herein as K-coils, referring to positively charged subunits whose charge is provided dominantly by lysine residues, and E-coils, referring to negatively charged subunits whose charge is provided dominantly by glutamic acid residues. Preferred examples from the above-mentioned application include SEQ ID NOS: 1-2. The K-coils and E-coils may also include repeats of K- and E-coils, respectively for use as heterodimer subunit peptides

Heterodimer-subunit peptides designed in accordance with the guidance presented in the above-referenced application typically show a preference for assembling in a parallel orientation versus an antiparallel orientation. For example, the exemplary peptides identified by SEQ ID NO:3 and SEQ ID NO:4 form parallel-configuration heterodimers as do other peptide sequences (as discussed in the WO 95/31480 application). When attaching a protein of interest to a first coil-forming peptide it is generally desirable to attach the protein of interest at the distal end of the heterodimer. In particular, where the heterodimer forms a parallel configuration, the second coil-forming peptide is preferably anchored to the substrate surface at its C-terminus, and the protein of interest is conjugated to the first coil-forming peptide at its N-terminus.

As noted, one of the two subunit peptides in the heterodimer is anchored to the substrate, and the other peptide contains a protein of interest intended to be presented. In both cases, the peptide can be synthesized or derivatized after synthesis, to provide the requisite attachment function. In general, most conjugating methods do not disrupt the coil-forming activity of either of the coil-forming peptide, nor do such conjugations disrupt the activity of the conjugated protein of interest.

Considering the modification of the second coil-forming peptide, the peptide may be synthesized at either its N- or C-terminus to carry additional terminal peptides that can function as a spacer between the substrate surface and the helical-forming part of the peptide. Alternatively, the second coil-forming peptide can be attached to the substrate surface through a high-affinity binding reaction such as between a biotin moiety carried on the peptide and an avidin molecule covalently attached to the surface.

The protein of interest can be synthesized by either solid-state, PCR, or recombinant methods, in vivo or in vitro to include the protein of interest at the end of the first coil-forming peptide that will orient distally in the assembled heterodimer. In forming the conjugate through solid-state methods, the protein of interest is preferably covalently attached to the N-terminal amino acid residue, or to one of the residues facing the exposed face of the heterodimer. Preferred coupling groups are the thiol groups of cysteine residues, which are easily modified by standard methods. Other useful coupling groups include thethioester of methionine, the imidazolyl group of histidine, the guanidinyl group of arginine, the phenolic group of tyrosine and the indolyl group of tryptophan. These coupling groups can be derivatized using reaction conditions known to those skilled in the art.

To bind the target protein-first coil-forming peptide conjugates 32, 33, 34 (FIG. 2) to the surface-immobilized second coil-forming peptide 30, the peptides are contacted under conditions that favor heterodimer formation. An exemplary medium favoring coiled-coil heterodimer formation is a physiologically-compatible aqueous solution typically having a pH of between about 6 and about 8 and a salt concentration of between about 50 mM and about 500 mM. Preferably, the salt concentration is between about 100 mM and about 200 mM. An exemplary benign medium has the following composition: 50 mM potassium phosphate, 100 mM KCl, pH 7. Equally effective media may be made by substituting, for example, sodium phosphate for potassium phosphate and/or NaCl for KCl. Heterodimers may form under conditions outside the above pH and salt range, medium, but some of the molecular interactions and relative stability of heterodimers vs. homodimers may differ from characteristics detailed above. For example, ionic interactions between the ionic groups that tend to stabilize heterodimers may break down at low or high pH values due to the protonation of, for example, Glu side chains at acidic pH, or the deprotonation of, for example, Lys side chains at basic pH. Such effects of low and high pH values on coiled-coil heterodimer formation may be overcome, however, by increasing salt concentration.

Increasing the salt concentration can neutralize the stabilizing ionic attractions or suppress the destabilizing ionic repulsions. Certain salts have greater efficacy at neutralizing the ionic interactions. For example, in the case of the K-coil peptide, a 1M or greater concentration of ClO⁴⁻ anions is required to induce maximal α-helical structure, whereas a 3M or greater concentration of Cl⁻ ions is required for the same effect. The effects of high salt on coiled-coil formation at low and high pH also show that interhelical ionic attractions are not essential for helix formation, but rather, control whether a coiled-coil tends to form as a heterodimer versus a homodimer. The E- and K-coil peptides can also be conjugated to proteins of interest or other biomolecules as in Example 2 of co-owned U.S. application Ser. No. 09/654,191 (Attorney Docket #: 4800-0015.31), which is expressly incorporated by reference herein in its entirety.

V. Applications

The invention includes, in one aspect, a method for carrying out the presentation of a plurality of target proteins. In general, a selected different-sequence nucleic acid molecule, from a plurality of different-sequence nucleic acid molecules is added to each of a plurality of wells in a substrate. Each well in the substrate has a first coil-forming peptide therein. Each different-sequence nucleic acid molecule has two portions: a common-sequence capture portion encoding a second coil-forming peptide, and a different-sequence target portion encoding a target protein.

The wells in the substrate are filled with a solution that contains protein synthesis components capable of expressing the different-sequence nucleic acid molecules under selected protein-synthesis conditions. The different-sequence nucleic acid molecules are then expressed. The target proteins expressed in each well bind to the well through coil-coil heterodimer formation with the substrate-bound coil forming peptide and are thus presented for analysis in the well in captured form. The wells can then be washed to remove unbound components.

In one embodiment, each different-sequence target portion is a different cDNA molecule selected from a library of cDNA molecules. Following expression, the presented proteins in each well have a different sequence 36, 37, 38, as illustrated in FIG. 2. The target proteins expressed in each well bind to the well through coil-coil heterodimer formation with the substrate-bound coil forming peptide and are thus presented for analysis in the well in captured form. Each protein is representative of the cDNA library. The presented proteins can then be screened against one or more drugs to identify the proteins that interact with a selected drug.

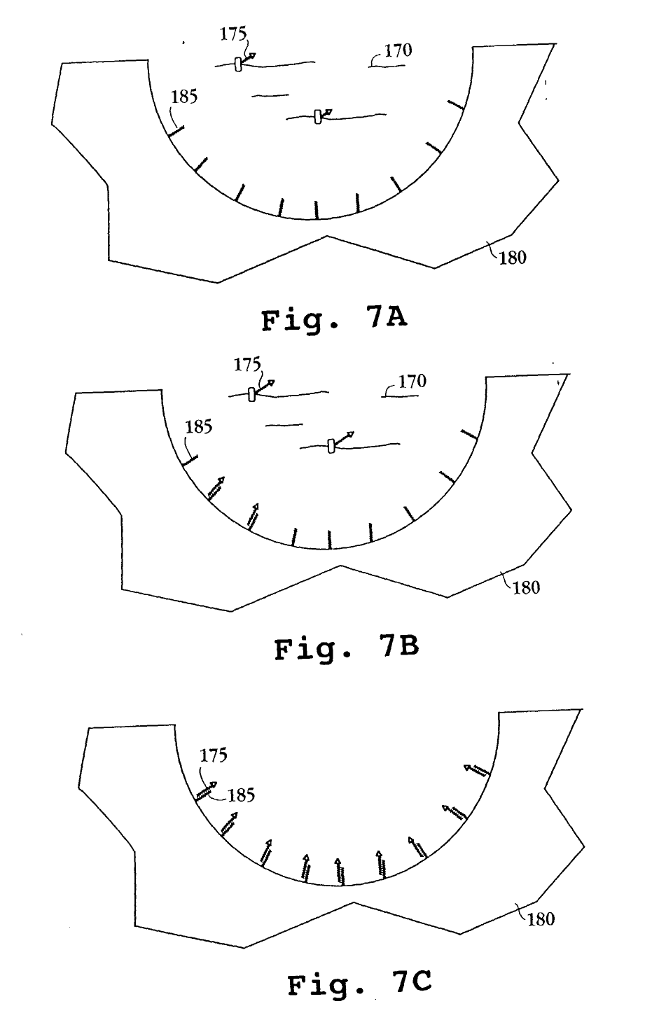

In another embodiment, as illustrated in FIG. 8, a protein 300 that has been mutated in a different region 302, 303 is placed in each well 310 such that on a given substrate 315 each well contains a different mutant 302 or 303 of the same protein 303. For example, a 96 well plate would have 96 different mutations of the same protein. A protein or drug is used to screen the plate for high affinity binding. Mass spectrometry is then used to identify where the mutation resides that is responsible for the increased binding affinity of the protein or drug.

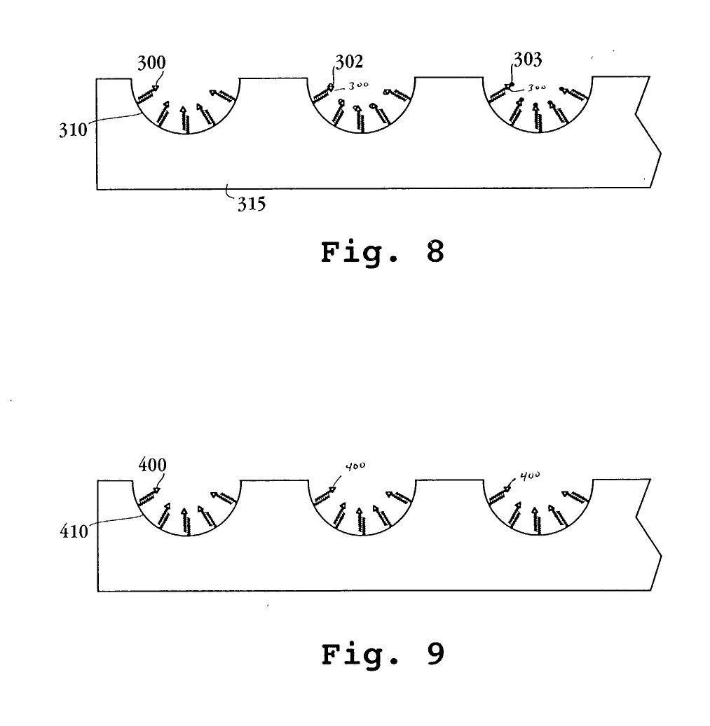

In yet another embodiment, each different-sequence target portion is encoded by the same DNA molecule. As illustrated in FIG. 9, following expression and protein binding, the presented proteins 400 in each well 410 are all be identical. The presented proteins can then be screened against a panel of different compounds to identify a drug that interacts with the presented protein. In one embodiment, a chemical library is subdivided into pools and then each pool is added to each well. Mass spectrometry is used to identify a compound or pool of compounds that bind specifically to the presented protein. In another embodiment a DNA library is subdivided into pools and then each pool is added to each well. Mass spectrometry is used to identify a specific DNA binding sequence for the presented protein. In yet another embodiment, the presented protein is an enzyme or enzyme variant that is presented in each well. A library of potential enzyme substrates are added to the wells, and mass spectrometry is used to identify product formation in the well; or in the case of an inhibitor, tight binding molecules.

The presented proteins can be used to monitor biochemical reactions as described above, such as, e.g., interactions of proteins, nucleic acids, small molecules, or the like. For example, the efficiency of specificity of interactions between antigens and antibodies; or of receptors (such as purified receptors or receptors bound to cell membranes) and their ligands, agonists or antagonists; and enzymes (such as proteases or kinases) and their substrates, or increases or decreases in the amount of substrate converted to a product; as well as many others. Such biochemical assays can be used to characterize properties of the target protein, or as the basis of a screening assay. For example, to screen samples for the presence of particular proteases Xa and VIIa), the samples can be assayed on combinations in which the target proteins are individual proteases. If a fluorogenic substrate specific for a particular presented protease binds to the protease and is cleaved, the substrate will fluoresce, usually as a result, e.g. of cleavage and separation between two energy transfer pairs, and the signal can be detected. In another example, to screen samples for the presence of a particular kinase(s) (e.g., Src, tyrosine kinase, or ZAP70), samples containing one or more kinases of interest can be assayed on combinations in which the bound, presented polypeptides can be selectively phosphorylated by one of the kinases of interest.

Using art-recognized, routinely determinable conditions, samples can be incubated with the array of substrates, in an appropriate buffer and with the necessary cofactors, for an empirically determined period of time. After treating (e.g., washing) each reaction under empirically determined conditions to remove unbound and undesired components, the bound components can be detected by mass spectrometry.

In another embodiment, the presented proteins can be used to screen for agents which modulate the interaction of a presented protein and a given probe. An agent can modulate the protein/probe interaction by interacting directly or indirectly with either the probe, the protein or a complex formed by the protein plus the probe. The modulation can take a variety of forms, including, but not limited to an increase or decrease in the binding affinity of the protein for the probe, an increase or decrease in the rate at which the protein and probe bind, a competitive or non-competitive inhibition of the binding of the probe to the protein, or an increase or decrease in the activity of the probe or the protein which can, in some cases, lead to an increase or decrease in the probe/protein interaction. Such agents can be synthetic or naturally-occurring substances. Also, such agents can be employed in their unaltered state or as aggregates with other species; and they can be attached, covalently or noncovaltently, to a binding member, either directly or via a specific binding substance.

From the foregoing, it can be seen how various objects and features of the invention are met.

TABLE 1

Sequences Provided In Support Of The Invention.

| | |
|---|---|
| Description | SEQ. ID NO. |

| | |
|---|---|
| E coil: | 1 |
| Glu Val Ser Ala Leu Glu Lys Glu Val Ser Ala Leu Glu Lys Glu Val Ser | |
| Ala Leu Glu Lys Glu Val Ser Ala Leu Glu Lys Glu Val Ser Ala Leu Glu | |
| Lys | |
| K coil: | 2 |
| Lys Val Ser Ala Leu Lys Glu Lys Val Ser Ala Leu Lys Glu Lys Val Ser | |
| Ala Leu Lys Glu Lys Val Ser Ala Leu Lys Glu Lys Val Ser Ala Leu Lys | |
| Glu | |
| Glu Val Glu Ala Leu Gln Lys Glu Val Ser Ala Leu GIu Lys Glu Val Ser | 3 |
| Ala Leu Glu Cys Glu Val Ser Ala Leu Glu Lys Glu Val Glu Ala Leu Gln | |
| Lys | |
| Lys Val Glu Ala Leu Lys Lys Lys Val Ser Ala Leu Lys GIu Lys Val Ser | 4 |
| Ala Leu Lys Cys Lys Val Ser Ala Leu Lys Glu Lys Val Glu Ala Leu Lys | |
| Lys | |
| T7 Promoter | 5 |
| TAATACGACTCACTATA | |
| T7 Terminator | 6 |
| CTAGCATAACCCCTTGGGGCCTCTAAACGGGTCTTGAGGGGTTTT | |
| TT | |

### VI. EXAMPLES

The following examples further illustrate the invention described herein and are in no way intended to limit the scope of the invention.

### Example 1

### In vitro Expression-MS Studies

A. Summary

The in vitro expression-mass spectrometry studies make use of MALDI-MS for the identification of proteins expressed in vitro. The objective of this study was to examine the feasibility of high-throughput protein analysis based on the coiled-coil protein presentation platform, in vitro protein expression and mass spectrometry. In general, E coil-tagged fusion proteins were cloned and expressed in a cell-free environment, and the expressed proteins were immediately captured by K coil peptides immobilized on 96-well ELISA plate. The captured E coil-tagged proteins were subsequently detected by MALDI-MS.

B. Experimental Details

The ribosomal extracts were either purchased from Promega Biosciences (San Luis Obispo, Calif.) or prepared in house from E. coli strain BL21*(DE3). Three DNA constructs such as the Tnl peptide-E coil (pMA2), Actin peptide-E coil (pS1A), and cMyc peptide-E coil (cMyc-E) were cloned into DNA plasmid pET-17b containing the T7 promoter sequence. Test in vitro expression of the 3 cloned plasmids showed that μg/ml level of proteins could be attained. The Costar sulfhydryl-bind 96-well plates were purchased from Corning Life Sciences (Acton, Mass.). K coil peptides were covalently immobilized onto the plate by incubating 1 μg/ml K coil-thiol in phosphate buffered saline (PBS, pH 6.5) containing 1 mM EDTA and 0.1 μM dithiothreitol in the wells for an hour at room temperature. Unreacted maleimide on the plate was blocked using 100 μM cysteine. 100 μl of in vitro expression reaction cocktails containing the cloned plasmid, S30 ribosomal extracts, buffer and required ingredients were added into the wells of K-coil immobilized plate and incubated at 37° C. for 2 hours. The plate was then washed 5 times with PBS (pH 7.4) containing 0.05% Tween-20 and 3 times with 10 mM phosphate buffer (no sodium chloride). The bound E coil-tagged proteins were extracted with 30 μl of 50% acetonitrile in 0.05% TFA/H₂O. 1 μl of the extract was added to the well of the MS target plate and mix with 1 μl of matrix solution (4 mg ferulic acid and 6 mg sinapinic acid in 1 ml of 50% acetonitrile in 0.05% TFA/H₂O). The mixture was allowed to crystallize at room temperature on a MALDI-MS target plate and finally analyzed with a Micromass TOF 2E mass spectrometer. The mass spectrometer was equipped with a 337 nm nitrogen laser and operated in linear positive-ion mode with an accelerating voltage of +20 KV. A multichannel plate high-mass detector was used for recording the spectra.

C. Results

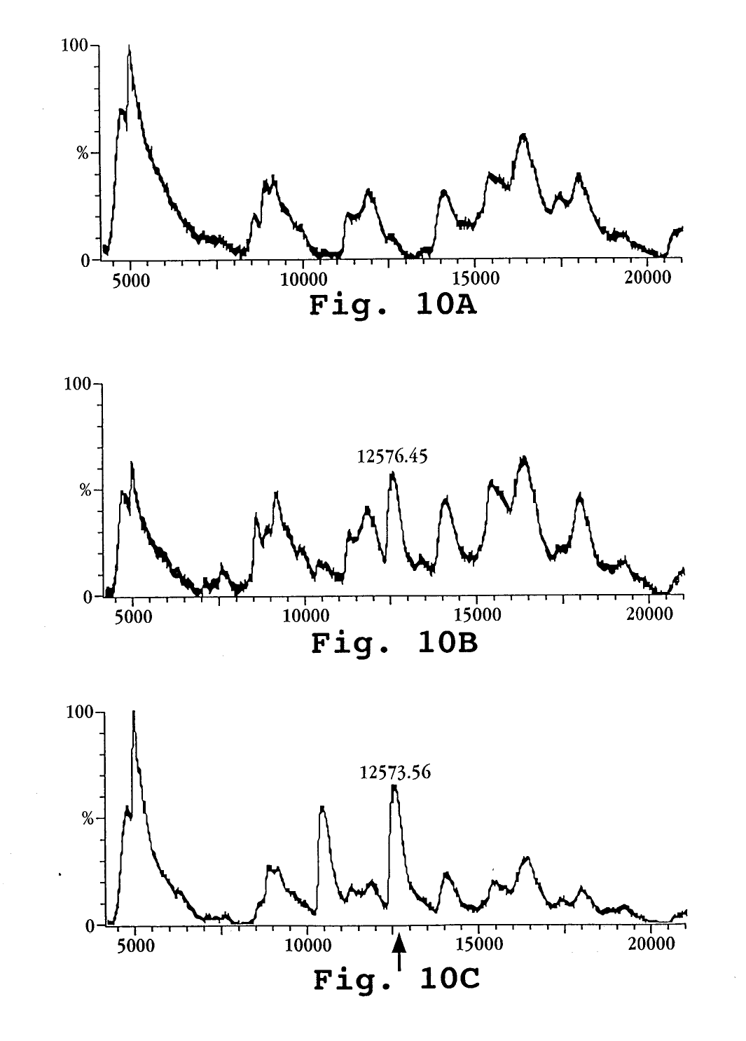

FIG. 10C shows the mass spectrum of Tnl peptide-E coil protein (pMA2) expressed using commercial extract and captured by K coil-immobilized surface. FIG. 10A is a negative control using plain pET-17b plasmid in the expression reaction cocktail. FIG. 10B shows non-specific binding of the fusion protein in cystine-immobilized surface. The peak corresponding to the mass of the fusion protein is indicated by an arrow in FIG. 10C.

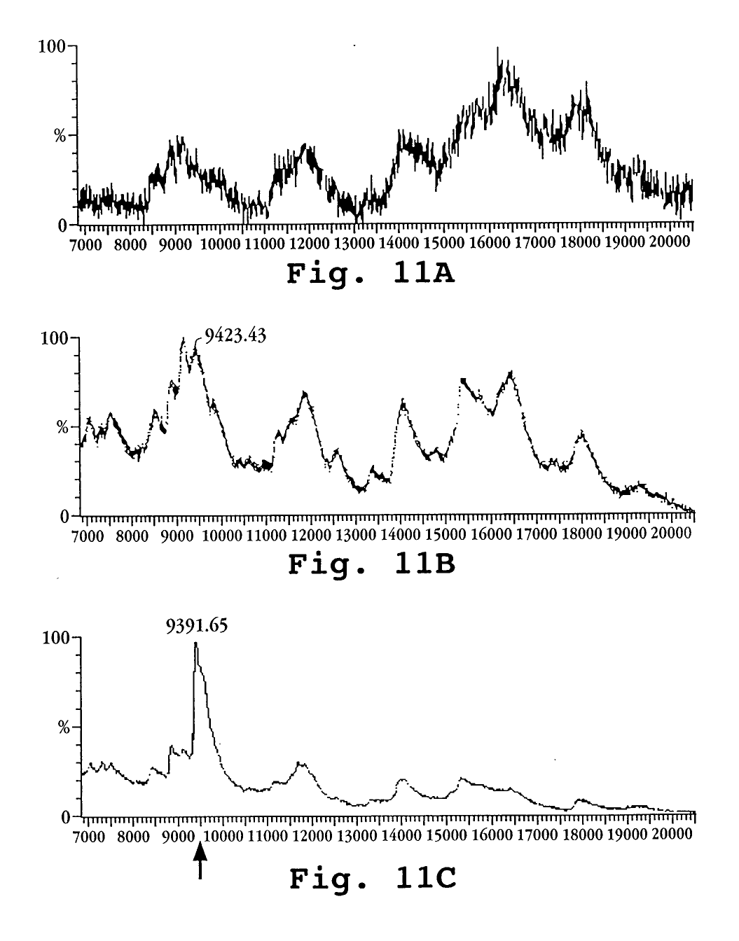

FIG. 11C shows the mass spectrum of Actin peptide-E coil protein (pS1A) expressed using commercial extract and captured by K coil-immobilized surface. FIG. 11A is a negative control using plain pET-17b plasmid in the expression reaction cocktail. FIG. 11B shows non-specific binding of the fusion protein in cystine-immobilized surface. The peak corresponding to the mass of the fusion protein is indicated by an arrow in FIG. 11C.

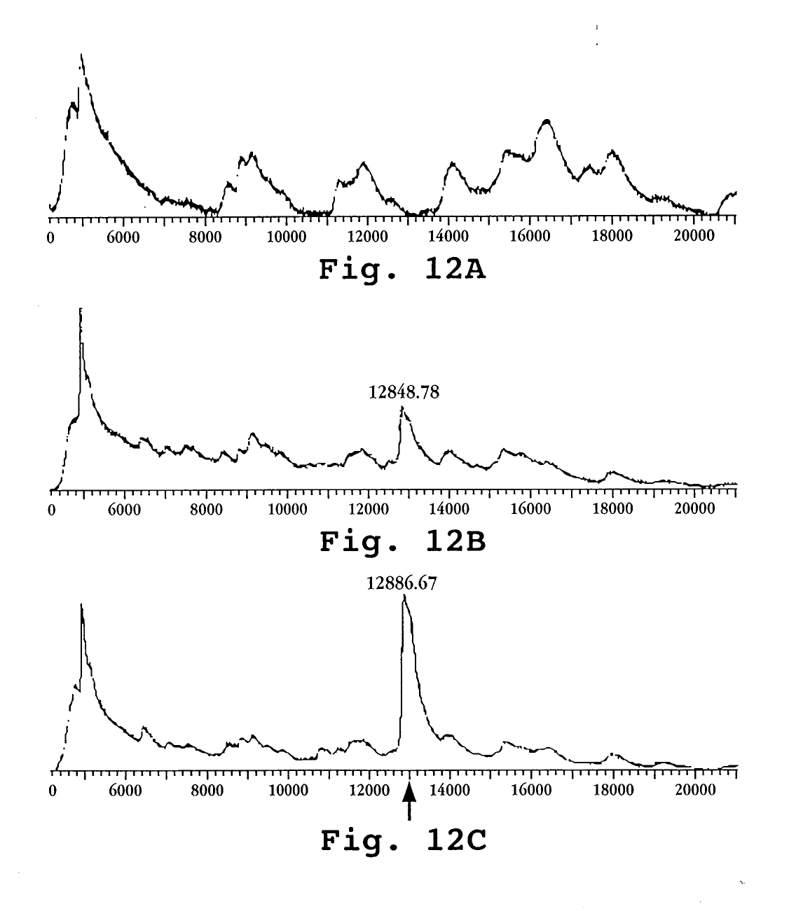

FIG. 12C shows the mass spectrum of cMyc peptide-E coil protein (cMyc-E) expressed using commercial extract and captured by K coil-immobilized surface. FIG. 12A is a negative control using plain pET-17b plasmid in the expression reaction cocktail. FIG. 12B shows non-specific binding of the fusion protein in cystine-immobilized surface. The peak corresponding to the mass of the fusion protein is indicated by an arrow in FIG. 12C.

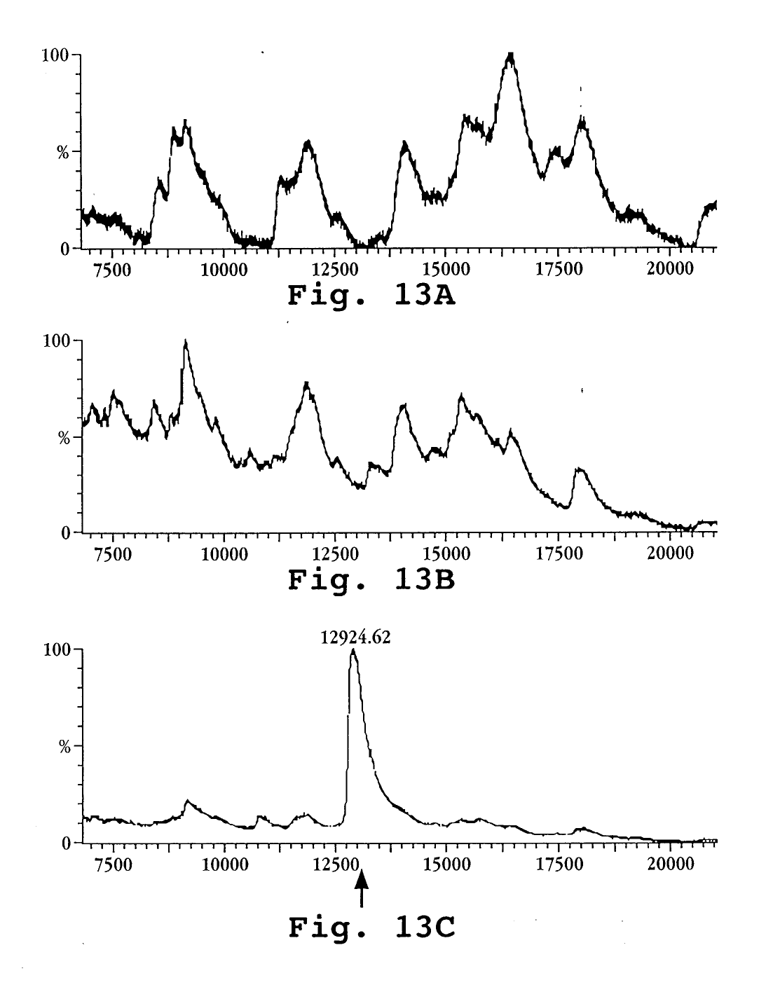

FIG. 13C shows the mass spectrum of cMyc peptide-E coil protein (cMyc-E) expressed using in house extract and captured by K coil-immobilized surface. FIG. 13A is a negative control using plain pET-17b plasmid in the expression reaction cocktail. FIG. 13B shows non-specific binding of the fusion protein in cystine-immobilized surface. The peak corresponding to the mass of the fusion protein is indicated by an arrow in FIG. 13C.

As shown in FIGS. 10-13, E coil-tagged proteins expressed from the three cloned plasmids were captured by the K coil immobilized plate and identified by MS (as indicated by the arrow in the figures). FIGS. 10-12 show results obtained from commercial ribosomal extracts as compared with the one using the in house extract (FIG. 13). The in house extract (FIG. 13) shows almost no non-specific binding.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the invention has been described with respect to particular embodiments, it will be apparent to those skilled in the art that various changes and modifications can be made without departing from the invention.

It is claimed:

1. A method for presenting a target protein for solid-phase analysis comprising
(a) forming, in a well on a substrate, a mixture containing
(i) a coding sequence comprising,
(A) a first nucleic acid sequence which encodes a first coil-forming peptide having a selected charge and being capable of interacting with a second, oppositely charged coil-forming peptide to form a stable α-helical coiled-coil heterodimer; and
(B) a second nucleic acid sequence encoding the target protein;
(ii) protein-synthesis components capable of expressing the target protein under selected protein-synthesis conditions in the well, said well having a surface which has been functionalized with the second coil-forming peptide;
(b) allowing the mixture to react under conditions such that the target protein is synthesized and binds to the well through coil-coil heterodimer formation, and is thus presented for analysis in the well in captured form; and
(c) washing said well to remove unbound components.

2. The method of claim 1, wherein said coding sequence is formed by cloning said second nucleic acid sequence into a cleavable site at which of a cloning vector containing said first nucleic acid sequence such that the first nucleic acid sequence is in frame with the second nucleic acid sequence.

3. The method of claim 2, wherein said cloning vector comprises in the 5′ to 3′ direction and operably linked
(a) a transcription and translation initiation region;
(b) the cleavable site at which a nucleic acid encoding the target protein can be inserted;
(c) said first nucleic acid sequence;
(d) a transcription and translation termination region.

4. The method of claim 2, wherein said cloning vector comprises in the 5′ to 3′ direction and operably linked
(a) a transcription and translation initiation region;
(b) said first nucleic acid sequence;
(c) the cleavable site at which a nucleic acid encoding the target protein can be inserted;
(d) a transcription and translation termination region.

5. The method of claim 1, wherein said coding sequence is formed by
(a) ligating the first nucleic acid sequence to the second nucleic acid sequence to form a chimeric coding sequence, and
(b) amplifying said chimeric coding sequence with PCR primers designed to hybridize with and amplify said chimeric coding sequence.

6. The method of claim 1, wherein said coding sequence is formed by
(a) optionally decapping said second nucleic acid sequence, where said second nucleic acid sequence is a mRNA molecule,
(b) ligating, to a 5′ end of the mRNA molecule to form a RNA template, a first oligonucleotide primer comprising
(i) said first nucleic acid sequence which encodes said first coil-forming peptide, and
(ii) a transcription initiation region which is oriented to transcribe towards the 3′ end,
(c) reverse transcribing the RNA template with reverse transcriptase, deoxyribonucleotide triphosphates and a second oligonucleotide primer comprising an oligonucleotide dT sequence to form first strand cDNA,
(d) removing the mRNA from the first strand cDNA,
(e) incubating the first strand cDNA, a DNA polymerase, deoxyribonucleotide triphosphates, and a third oligonucleotide primer comprising at least 12 nucleotides of the first primer sequence, to form double stranded cDNA,
(f) amplifying the double stranded cDNA with DNA polymerase, deoxyribonucleotide triphosphates, a fourth oligonucleotide primer complementary to at least 12 nucleotides of the 3′ end of a first strand of the cDNA, and a fifth oligonucleotide primer complementary to at least 12 nucleotides of the 3′ end of the second strand.

7. The method of claim 1, wherein said coding sequence is formed by
(a) optionally decapping said second nucleic acid sequence, where said second nucleic acid sequence is a mRNA molecule,
(b) ligating a first oligonucleotide primer to a 5′ end of the mRNA molecule to form a RNA template,
(c) reverse transcribing the RNA template with reverse transcriptase, deoxyribonucleotide triphosphates and a first oligonucleotide primer comprising an oligonucleotide dT sequence to form first strand cDNA,
(d) removing the mRNA from the first strand cDNA,
(e) incubating the first strand cDNA, a DNA polymerase, deoxyribonucleotide triphosphates, and a second oligonucleotide primer comprising at least 12 nucleotides of the first primer sequence, to form double stranded cDNA,
(f) amplifying the double stranded cDNA with DNA polymerase, deoxyribonucleotide triphosphates, a third oligonucleotide primer comprising
(i) a region complementary to at least 12 nucleotides of the 3′ end of a first strand of the cDNA, and
(ii) a restriction enzyme site compatible with a first restriction enzyme site in a cloning vector,
and a fourth oligonucleotide primer comprising
(i) a region complementary to at least 12 nucleotides of the 3′ end of the second strand, and
(ii) a restriction enzyme site compatible with a second restriction enzyme site in said cloning vector;
(g) digesting said amplification product and said cloning vector with restriction enzymes capable of cutting at said first and second restriction enzyme sites; and
(h) cloning said digested amplification product into said cloning vector.

8. The method of claims 5 and 6, wherein said amplification product is translated in vitro, by further including the steps of
(a) transcribing the template sequence in vitro using a DNA-dependent RNA polymerase that recognizes the transcription initiation region in said amplification product; and
(b) combining the transcription products with an appropriate cell free in vitro translation system.

9. The method of claim 2 and 7, wherein said nucleic acid sequence of interest is translated in vitro, by further including the steps of
(a) linearizing the cloning vector with a restriction enzyme that cleaves downstream from the coding sequence;
(b) transcribing the template sequence in vitro using a DNA-dependent RNA polymerase that recognizes the transcription initiation region in said cloning vector; and
(c) combining the transcription products with an appropriate cell free in vitro translation system.

10. The method of claim 1, wherein said coding sequence is transformed or transfected into cells capable of translating said coding sequence, where said protein-synthesis components comprise said cells.

11. A method for carrying out the presentation of a plurality of target proteins, comprising
(a) adding to each of a plurality of wells in a substrate, each well having a first coil-forming peptide therein, a selected one of a plurality of different-sequence nucleic acid molecules, each having a common-sequence capture portion encoding a second coil-forming peptide and a different-sequence target portion encoding a target protein;
(b) filling said wells with a solution comprising protein synthesis components capable of expressing the different-sequence nucleic acid molecules under selected protein-synthesis conditions;
(c) promoting expression of said different-sequence nucleic acid molecules under such conditions, wherein the target protein expressed in each well binds to the well through coil-coil heterodimer formation and is thus presented for analysis in the well in captured form; and
(d) washing the wells to remove unbound components.

12. The method of claim 11, wherein said substrate is an array of 96 wells.

13. The method of claim 11, wherein said substrate is a MALDI-MS plate having wells capable of holding said solution.

14. A kit for presenting one or more target proteins for solid-phase analysis for use with a cell free in vitro translation system, comprising
(a) a substrate containing a plurality of wells, wherein each well is functionalized with a first coil-forming peptide having a selected charge and being capable of interacting with a second, oppositely charged coil-forming peptide to form a stable α-helical coiled-coil heterodimer;
(b) a cloning vector comprising in the 5′ to 3′ direction and operably linked
(i) a transcription and translation initiation region,
(ii) a nucleic acid sequence which encodes said second coil-forming peptide,
(iii) a transcription and translation termination region;
(c) said vector also having a cleavable site at which a nucleic acid encoding a heterologous protein can be inserted between (i) and (ii) or between (ii) and (iii).

15. A multiplexed in vitro cell free protein synthesis system, comprising
(a) a substrate comprising a plurality of wells, each well having bound thereto a first coil-forming peptide having a selected charge and being capable of interacting with a second, oppositely charged coil-forming peptide to form a stable α-helical coiled-coil heterodimer;
(b) contained in each of said wells,
(i) a coding sequence comprising,
(A) a first nucleic acid sequence which encodes a first coil-forming peptide having a selected charge and being capable of interacting with a second, oppositely charged coil-forming peptide to form a stable α-helical coiled-coil heterodimer; and
(B) a second nucleic acid sequence encoding the target protein;
(ii) protein-synthesis components capable of expressing the target protein under selected protein-synthesis conditions in the well, said well having a surface which has been functionalized with the second coil-forming peptide;
whereby the mixture reacts under conditions such that the target protein is synthesized and binds to the well through coil-coil heterodimer formation, and is thus presented for analysis in each of the wells in captured form.


SEQUENCE LISTING (6 sequences)

SEQ ID NO: 1 - protein, 35 aa, Artificial Sequence
  E coil-forming peptide
EVSALEKEVSALEKEVSALEKEVSALEKEVSALEK

SEQ ID NO: 2 - protein, 35 aa, Artificial Sequence
  K coil-forming peptide
KVSALKEKVSALKEKVSALKEKVSALKEKVSALKE

SEQ ID NO: 3 - protein, 35 aa, Artificial Sequence
  coil-forming peptide
EVEALQKEVSALEKEVSALECEVSALEKEVEALQK

SEQ ID NO: 4 - protein, 35 aa, Artificial Sequence
  coil-forming peptide
KVEALKKKVSALKEKVSALKCKVSALKEKVEALKK

SEQ ID NO: 5 - DNA, 17 bp, Artificial Sequence
  T7 promoter
taatacgactcactata

SEQ ID NO: 6 - DNA, 47 bp, Artificial Sequence
  T7 terminator
ctagcataaccccttggggcctctaaacgggtcttgaggggtttttt